SEQUENCE LISTING (2 sequences)

SEQ ID NO: 1 - DNA, 1305 bp, Escherichia coli
  source 1..1305: mol_type = other DNA
atgaaaacccgtacacaacaaattgaagaattacagaaagagtggactcaaccgcgttgggaaggcattactcgcccatacagtgcggaagatgtggtgaaattacgcggttcagtcaatcctgaatgcacgctggcgcaactgggcgcagcgaaaatgtggcgtctgctgcacggtgagtcgaaaaaaggctacatcaacagcctcggcgcactgactggcggtcaggcgctgcaacaggcgaaagcgggtattgaagcagtctatctgtcgggatggcaggtagcggcggacgctaacctggcggccagcatgtatccggatcagtcgctctatccggcaaactcggtgccagctgtggtggagcggatcaacaacaccttccgtcgtgccgatcagatccaatggtccgcgggcattgagccgggcgatccgcgctatgtcgattacttcctgccgatcgttgccgatgcggaagccggttttggcggtgtcctgaatgcctttgaactgatgaaagcgatgattgaagccggtgcagcggcagttcacttcgaagatcagctggcgtcagtgaagaaatgcggtcacatgggcggcaaagttttagtgccaactcaggaagctattcagaaactggtcgcggcgcgtctggcagctgacgtgacgggcgttccaaccctgctggttgcccgtaccgatgctgatgcggcggatctgatcacctccgattgcgacccgtatgacagcgaatttattaccggcgagcgtaccagtgaaggcttcttccgtactcatgcgggcattgagcaagcgatcagccgtggcctggcgtatgcgccatatgctgacctggtctggtgtgaaacctccacgccggatctggaactggcgcgtcgctttgcacaagctatccacgcgaaatatccgggcaaactgctggcttataactgctcgccgtcgttcaactggcagaaaaacctcgacgacaaaactattgccagcttccagcagcagctgtcggatatgggctacaagttccagttcatcaccctggcaggtatccacagcatgtggttcaacatgtttgacctggcaaacgcctatgcccagggcgagggtatgaagcactacgttgagaaagtgcagcagccggaatttgccgccgcgaaagatggctataccttcgtatctcaccagcaggaagtgggtacaggttacttcgataaagtgacgactattattcagggcggcacgtcttcagtcaccgcgctgaccggctccactgaagaatcgcagttctaa

SEQ ID NO: 2 - DNA, 1602 bp, Escherichia coli
  source 1..1602: mol_type = other DNA
atgactgaacaggcaacaacaaccgatgaactggctttcacaaggccgtatggcgagcaggagaagcaaattcttactgccgaagcggtagaatttctgactgagctggtgacgcattttacgccacaacgcaataaacttctggcagcgcgcattcagcagcagcaagatattgataacggaacgttgcctgattttatttcggaaacagcttccattcgcgatgctgattggaaaattcgcgggattcctgcggacttagaagaccgccgcgtagagataactggcccggtagagcgcaagatggtgatcaacgcgctcaacgccaatgtgaaagtctttatggccgatttcgaagattcactggcaccagactggaacaaagtgatcgacgggcaaattaacctgcgtgatgcggttaacggcaccatcagttacaccaatgaagcaggcaaaatttaccagctcaagcccaatccagcggttttgatttgtcgggtacgcggtctgcacttgccggaaaaacatgtcacctggcgtggtgaggcaatccccggcagcctgtttgattttgcgctctatttcttccacaactatcaggcactgttggcaaagggcagtggtccctatttctatctgccgaaaacccagtcctggcaggaagcggcctggtggagcgaagtcttcagctatgcagaagatcgctttaatctgccgcgcggcaccatcaaggcgacgttgctgattgaaacgctgcccgccgtgttccagatggatgaaatccttcacgcgctgcgtgaccatattgttggtctgaactgcggtcgttgggattacatcttcagctatatcaaaacgttgaaaaactatcccgatcgcgtcctgccagacagacaggcagtgacgatggataaaccattcctgaatgcttactcacgcctgttgattaaaacctgccataaacgcggtgcttttgcgatgggcggcatggcggcgtttattccgagcaaagatgaagagcacaataaccaggtgctcaacaaagtaaaagcggataaatcgctggaagccaataacggtcacgatggcacatggatcgctcacccaggccttgcggacacggcaatggcggtattcaacgacattctcggctcccgtaaaaatcagcttgaagtgatgcgcgaacaagacgcgccgattactgccgatcagctgctggcaccttgtgatggtgaacgcaccgaagaaggtatgcgcgccaacattcgcgtggctgtgcagtacatcgaagcgtggatctctggcaacggctgtgtgccgatttatggcctgatggaagatgcggcgacggctgaaatttcccgtacctcgatctggcagtggatccatcatcaaaaaacgttgagcaatggcaaaccggtgaccaaagccttgttccgccagatgctgggcgaagagatgaaagtcattgccagcgaactgggcgaagaacgtttctcccaggggcgttttgacgatgccgcacgcttgatggaacagatcaccacttccgatgagttaattgatttcctgaccctgccaggctaccgcctgttagcgtaa

(19) United States
(12) Patent Application Publication
(10) Pub. No.: US 2026/0250723 A1
(43) Pub. Date: Aug. 27, 2026

(54) GENETICALLY ENGINEERED MICROORGANISM AND METHOD FOR PRODUCING SUCCINATE AND METHOD FOR MAKING GENETICALLY ENGINEERED MICROORGANISM

(71) Applicant: National Yang Ming Chiao Tung University, Hsinchu City (TW)

(72) Inventors: Ethan I. LAN, Hsinchu City (TW); Hsin Hsien YU, New Taipei City (TW); You-An CHEN, Hsinchu County (TW)

(21) Appl. No.: 19/176,208

(22) Filed: Apr. 11, 2025

(30) Foreign Application Priority Data
Feb. 25, 2025 (TW) 114106993

(51) Int. Cl. C12P 7/46; C12N 9/02; C12N 9/04; C12N 9/10; C12N 9/88; C12N 15/52; C12N 15/74

(52) U.S. Cl. CPC C12P 7/46; C12N 9/0006; C12N 9/001; C12N 9/1003; C12N 9/1025; C12N 9/88; C12N 15/52; C12N 15/74; C12Y 101/01036; C12Y 101/01244; C12Y 103/01085; C12Y 103/05001; C12Y 201/02001; C12Y 203/03001; C12Y 203/03009; C12Y 401/03001

(57) ABSTRACT

A genetically engineered microorganism for producing succinate, including: a host cell derived from a methylotrophic bacterium. The host cell has an ethylmalonyl-CoA pathway. The activity of succinate dehydrogenase in the methylotrophic bacterium is reduced or inactivated, and the genetically engineered microorganism is capable of growing using methanol as the sole carbon source and producing succinate. Also provided herein are methods for producing succinate and methods for making genetically engineered microorganisms for producing succinate.

### CROSS-REFERENCE TO RELATED APPLICATION

This application claims priority to Taiwan Application Serial Number 114106993, filed Feb. 25, 2025, which is herein incorporated by reference in its entirety.

The Sequence Listing associated with this application is filed in electronic format via EFS-Web and is hereby incorporated by reference into the specification in its entirety. The name of the text file containing the Sequence Listing is NP-38940-US_SEQ_LIST.txt. The size of the text file is 6 KB, and the text file was created on Apr. 11, 2025.

### BACKGROUND

### Field of Invention

The present disclosure relates to genetically engineered microorganisms for biosynthetically producing succinate, methods for producing succinate, and methods for making genetically engineered microorganisms for producing succinate.

### Description of Related Art

Succinate (also known as butanedioic acid or succinic acid) is a colorless crystal with a wide range of industrial applications, such as a chemical intermediate in the production of varnishes and perfumes, and as a flavoring agent, antimicrobial agent, or neutralizer in the food industry. It also has specific applications in the chemical markets for the production of coatings, surfactants, dyes, detergents, green solvents, biodegradable plastics, and components that promote the growth of plants and animals. Additionally, succinate can be converted into other bulk chemicals, such as γ-butyrolactone, tetrahydrofuran, adipic acid, and 1,4-butanediol, a major precursor for tire production.

Traditional chemical methods for producing succinate include paraffin oxidation, catalytic hydrogenation, and electroreduction. Paraffin oxidation involves the oxidation of paraffin in the presence of calcium or manganese catalysts to generate a mixture of carboxylic acids, followed by separation steps such as steam distillation and crystallization to obtain succinate. Catalytic hydrogenation involves the hydrogenation of maleic anhydride or fumaric acid in the presence of hydrogen and a nickel or noble metal catalyst to produce succinate. Electroreduction involves the electrolysis of phthalic anhydride, sulfuric acid, and water in a ceramic electrolytic cell to form succinate.

However, these methods generally have the following issues: (1) the reaction conditions are harsh and the procedures are complex, requiring high temperature and pressure environments; (2) the catalysts used (e.g., hydrogen) are hazardous; and (3) reliance on petrochemical raw materials, such as paraffin and maleic anhydride, which are petroleum derivatives and are not environmentally friendly.

Therefore, there is a need to find green and environmentally friendly biotechnological methods to produce succinate as an alternative to traditional chemical methods.

### SUMMARY

Embodiments of the present disclosure provide methods for producing succinate by biosynthesis and developing related genetically engineered microorganisms.

Since the use of carbon sources such as sugars or glycerol for fermentative production of succinate raises concerns about competition with food resources and the use of petrochemical resources, embodiments of the present disclosure develop genetically engineered microorganisms that use methanol as a carbon source for the biosynthetic production of succinate.

Embodiments of the present disclosure provide a genetically engineered microorganism for producing succinate, including a host cell derived from a methylotrophic bacterium. The host cell has an ethylmalonyl-CoA pathway, the activity of succinate dehydrogenase in the methylotrophic bacterium is reduced or inactivated, and the genetically engineered microorganism is capable of growing and producing succinate using methanol as the sole carbon source.

In some embodiments, the genetically engineered microorganism for producing succinate further includes: at least one heterologous gene encoding an enzyme of the glyoxylate cycle or an enzyme that converts isocitrate to glyoxylate and succinate.

In some embodiments, the at least one heterologous gene is located in a plasmid.

In some embodiments, the at least one heterologous gene includes: a gene encoding isocitrate lyase (aceA), a gene encoding malate synthase (aceB), or a combination thereof.

In some embodiments, the genetically engineered microorganism is obtained through adaptive laboratory evolution.

In some embodiments, during the adaptive laboratory evolution, performing serial passage using a mineral medium having methanol as a sole carbon source.

In some embodiments, the genetically engineered microorganism is a strain of Methylobacterium extorquens Otter-1, with deposit number BCRC 911257.

In some embodiments, the genetically engineered microorganism has: enhanced expression of at least one gene involved in methanol metabolism pathway; enhanced expression of at least one gene involved in the serine cycle; reduced expression of at least one gene involved in the tricarboxylic acid (TCA) cycle pathway; and enhanced expression of at least one gene involved in the ethylmalonyl-CoA pathway.

In some embodiments, the at least one gene involved in the methanol metabolism pathway includes a methanol dehydrogenase gene (mxaF); the at least one gene involved in the serine cycle includes a serine hydroxymethyltransferase gene (glyA); the at least one gene involved in the TCA cycle pathway includes a citrate synthase gene (gltA); and the at least one gene involved in the ethylmalonyl-CoA pathway includes: an acetoacetyl-CoA reductase gene (phaB), a crotonyl-CoA carboxylase/reductase gene (ccr), or a combination thereof.

Embodiments of the present disclosure provide a method for producing succinate, including: cultivating a strain for producing succinate, including: cultivating the genetically engineered microorganism for producing succinate in a mineral medium, wherein the sole carbon source in the mineral medium is methanol.

In some embodiments, the cultivating of the strain for producing succinate is performed using fed-batch fermentation or continuous batch fermentation.

In some embodiments, the method for producing succinate further includes: supplementing a nitrogen source to the mineral medium.

In some embodiments, the nitrogen source is (NH₄)₂NO₃ or NH₄OH.

In some embodiments, the mineral medium comprises vitamin B₁₂.

In some embodiments, the mineral medium comprises NaCl, and a concentration of the NaCl is less than 5 g/L.

In some embodiments, the mineral medium comprises NaCl and NH₄OH as neutralizers.

Embodiments of the present disclosure provide a method for making a genetically engineered microorganism, including: performing a first gene editing operation to reduce or inactivate the activity of succinate dehydrogenase (Sdh) in a methylotrophic bacterium, wherein the methylotrophic bacterium comprises an ethylmalonyl-CoA pathway; and performing a second gene editing operation or an adaptive evolution operation to obtain a strain for producing succinate using methanol as the sole carbon source.

In some embodiments, through the second gene editing operation, the strain for producing succinate expresses a heterologous enzyme of the glyoxylate cycle or an enzyme that converts isocitrate to glyoxylate and succinate.

In some embodiments, through the second gene editing operation or the adaptive evolution operation, the strain for producing succinate has increased methanol metabolism pathway activity, reduced TCA cycle pathway activity, and increased carbon flux through the ethylmalonyl-CoA pathway.

In some embodiments, the adaptive evolution operation comprises serial passaging the methylotrophic microorganism using a mineral medium having methanol as a sole carbon source.

### BRIEF DESCRIPTION OF THE DRAWINGS

Aspects of the present disclosure are best understood from the following detailed description when read with the accompanying figures.

FIG. 1 illustrates a biosynthetic pathway from methanol to succinate according to some embodiments of the present disclosure.

FIG. 2 illustrates a central metabolic map of a methylotrophic bacterium according to some embodiments.

FIG. 3 is a bar graph showing the growth of strains in accordance with an experimental example.

FIG. 4A is a curve graph showing the growth and succinate titer of bacteria strains in accordance with an experimental example.

FIG. 4B is a curve graph showing the growth of bacteria strains in accordance with an experimental example.

FIG. 4C is a curve graph showing the succinate titer of bacteria strains in accordance with an experimental example.

FIG. 5 is a bar graph showing the effects of adding different compounds on the growth and succinate titer of a bacteria strain in accordance with an experimental example.

FIG. 6A illustrates a central metabolic map of a genetically engineered microorganism according to some embodiments.

FIG. 6B shows the design of a plasmid according to an experimental example.

FIG. 6C shows the design of a plasmid according to an experimental example.

FIG. 6D is a bar graph showing the growth and succinate titer of the bacteria strain transformed with the plasmids respectively from FIGS. 6B and 6C and in mineral medium containing methanol.

FIG. 7A illustrates the operation flow of adaptive laboratory evolution in accordance with an experimental example.

FIG. 7B is a curve graph showing the growth of different bacteria strains obtained through adaptive laboratory evolution in accordance with an experimental example.

FIG. 8 is a bar graph showing the expression of some genes in a strain obtained through adaptive laboratory evolution and using methanol as the sole carbon source in accordance with an experimental example.

FIG. 9 is a curve graph showing the growth and succinate titer of a bacteria strain using methanol as the sole carbon source in accordance with an experimental example.

FIG. 10 is a curve graph showing the growth and succinate titer of a strain during fed-batch fermentation using methanol as the sole carbon source in accordance with an experimental example.

FIGS. 11A to 11C are curve graphs showing the growth of a bacteria strain in accordance with an experimental example. FIGS. 11A and 11B show the effects of different concentrations of CoCl₂ in the medium on the growth of the bacteria strain. FIG. 11C shows the effects of adding 4 μM CoCl₂ and 4 μM vitamin B complex to the medium on the growth of the bacteria strain.

FIGS. 11D to 11F are curve graphs showing the succinate titers under the test conditions shown in FIGS. 11A to 11C, respectively.

FIG. 12 shows a curve graph showing the toxicity of different sodium ion concentrations on the bacteria strain in accordance with an experimental example.

FIGS. 13A and 13B are curve graphs showing the growth curves and the succinate titers of the bacteria strain when NaOH, KOH, and NH₄OH were used as neutralizers in the culture in accordance with an experimental example.

FIG. 14 is a bar graph showing the residual methanol concentrations after culturing for 12 hours in different fermentation shake flasks covered with different caps in accordance with an experimental example.

FIGS. 15A to 15C are curve graphs showing methanol concentration changes, succinate titers, and strain growths, respectively, in different fermentation shake flasks covered with different caps in accordance with an experimental example.

### DETAILED DESCRIPTION

The following will use detailed descriptions and drawings to clearly illustrate the spirit of this disclosure. It should be understood that the present disclosure can be varied in different aspects without departing from the scope of the present disclosure, and the description and drawings therein are for illustration, not for limitation.

The strain of Methylobacterium extorquens Otter-1 of present disclosure has been deposited at NITE Patent Microorganisms Depositary (NPMD) in Japan (address of depositary institution: #122, 2-5-8 Kazusakamatari, Kisarazu-shi, Chiba 2920818, Japan). The strain of Methylobacterium extorquens Otter-1 of present disclosure also has been deposited at Bioresource Collection and Research Center (BCRC) in Taiwan (address of depositary institution: No. 331 Shih-Pin Road, Hsinchu, Taiwan 300) on Dec. 23, 2024, and the deposit number is BCRC 911257.

Currently, there are three main pathways for microbial synthesis of succinate. (1) Using the natural reductive tricarboxylic acid (TCA) cycle pathway (also known as the fermentation pathway) in anaerobic conditions, wherein phosphoenolpyruvate (PEP) is sequentially converted to oxaloacetate (OAA), malate, fumarate, and finally succinate. (2) Using the natural oxidative TCA cycle pathway in aerobic conditions, wherein acetyl-CoA and OAA are synthesized into citrate, which is then converted to isocitrate, α-ketoglutarate, succinyl-CoA, and finally succinate. (3) Using the glyoxylate shunt pathway in aerobic conditions, wherein isocitrate lyase (AceA) is expressed to cleave isocitrate into glyoxylate and succinate, and glyoxylate is then synthesized into malate with acetyl-CoA via malate synthase (AceB).

Previous technologies use naturally succinate-producing strains, such as Actinobacillus succinogenes, Anaerobiospirillum succiniciproducens, Mannheimia succiniciproducens, Clostridium thermossuccinogenes, and Cytophaga succinicans, to produce succinate using sugars or glycerol as carbon sources. Additionally, previous technologies use genetic engineering techniques to modify common model organisms, such as E. coli or Saccharomyces cerevisiae, to produce succinate using sugars or glycerol as carbon sources. However, the drawback of these methods is that the use of such carbon sources for fermentation may compete with food resources and raise concerns about the use of petrochemical resources.

Therefore, in various embodiments of the present disclosure, another renewable resource, methanol, is used as the carbon source for the bioconversion of succinate.

Methanol is an electron-rich organic C1 carbon source. Traditionally, methanol is mainly derived from coal, natural gas, and biomass. Recently, with the industrialization of carbon dioxide hydrogenation technology for producing green methanol, methanol has become a sustainable, environmentally friendly, and renewable emerging raw material. Additionally, methanol can also be obtained from methane, giving methanol many flexible sources. It is estimated that by 2030, global methanol production capacity will increase to 300 million tons. Therefore, using methanol as a non-food-based fermentation raw material for the microbial synthesis of succinate can reduce the succinate market's dependence on petrochemical raw materials and sugars, while also enhancing the reuse value of carbon dioxide in conjunction with the development of green methanol.

Various embodiments of the present disclosure utilize metabolic engineering, synthetic biology, and microbiology technologies to design non-naturally occurring microorganisms for the biosynthesis of succinate from methanol. Metabolic engineering improves the production efficiency and sustainability of high-value compounds by modifying microbial metabolic pathways. Synthetic biology applies gene editing and synthetic genomics technologies to achieve specific biosynthetic goals. Microbiology studies microbial growth and metabolism, providing theoretical and experimental support for these technologies.

As used herein, the term “endogenous” refers to the activity of a nucleic acid sequence or gene product naturally present in the host microorganism.

As used herein, the term “exogenous” refers to the activity of a nucleic acid sequence or gene product introduced into the host microorganism.

As used herein, the term “homologous” refers to a nucleic acid sequence or gene product derived from the host microorganism.

As used herein, the term “heterologous” refers to a substance that is not naturally present in the host cell. For example, a nucleic acid sequence is considered heterologous to the host cell if at least one of the following is true: (a) the nucleic acid is not naturally found in that cell (i.e., it is an “exogenous” nucleic acid); (b) the nucleic acid is naturally found in the given host cell (i.e., it is “endogenous”), but the nucleic acid or the RNA or protein produced by its transcription and translation is produced or present in the host cell in a non-natural amount (e.g., greater or less than naturally occurring); (c) the nucleic acid encodes an endogenous protein of the host cell but differs in sequence from the endogenous nucleotide sequence encoding the same protein (with the same or substantially the same amino acid sequence), typically resulting in the protein being produced in greater amounts in the cell, or in the case of an enzyme, producing a mutated form with altered (e.g., higher, lower, or different) activity; and/or (d) the nucleic acid contains two or more nucleotide sequences that are not naturally found in the same relationship to each other in the host cell.

As used herein, enhanced or reduced gene expression in a genetically engineered microorganism refers to the gene expression relative to a strain of the microorganism that has not undergone a specific genetic engineering. The enhancement in enzyme activity refers to at least a 150% increase in protein activity compared to an appropriate control strain. The reduction in enzyme product activity refers to a decrease of at least 75% in enzyme activity compared to microbial strains that have not undergone specific genetic engineering operation. Preferably, a reduction in activity of at least 80%, 85%, 90%, 95% is achieved, and in a more preferred embodiment, the activity is eliminated (100%).

In embodiments, the regulation and optimization of transcription, translation, protein stability, protein function, or the like may be achieved through the use of genetic engineering and appropriate culture conditions. In embodiments, the optimization of metabolic flux may include reducing, disrupting, or knocking out competitive metabolic pathways that compete with intermediates leading to the desired pathway. In some embodiments, some of the multiple genes involved in the succinate biosynthesis are endogenous. In some embodiments, some of the genes involved in succinate biosynthesis are foreign to the host or have been modified through mutagenesis, recombination, and/or association with heterologous expression control sequences in the endogenous host cell, and thus may be heterologous to the host microorganism.

In embodiments of the present disclosure, methanol-utilizing microorganisms, such as methylotrophic bacteria, which have a natural ethylmalonyl-CoA (EMCP) synthesis pathway, are used to metabolize methanol and produce succinate. In some embodiments, the host of the genetically engineered microorganism is derived from methylotrophic bacteria, such as Methylobacterium, Methylophilus, Methylomonas, or the like.

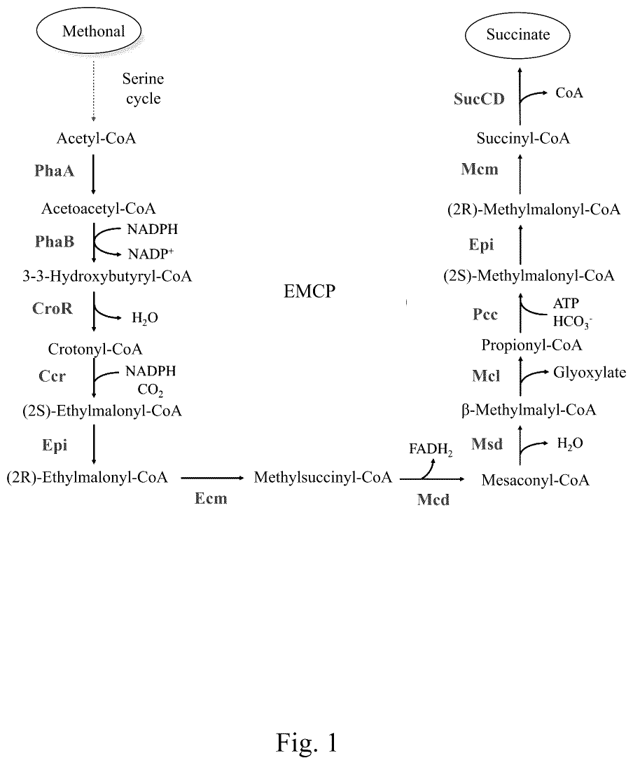

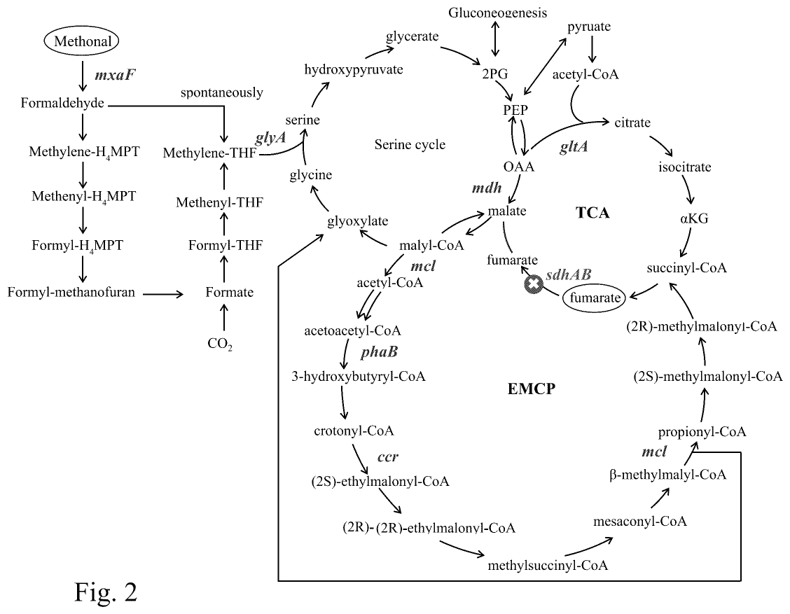

Referring to FIGS. 1 and 2, FIG. 1 illustrates the metabolic pathway from acetyl-CoA to succinate production. FIG. 2 shows a schematic diagram of the central metabolism of a methylotrophic bacterium, including methanol metabolism pathway, serine cycle pathway, tricarboxylic acid (TCA) cycle pathway, and ethylmalonyl-CoA pathway.

In the metabolic pathway of methylotrophic bacteria, methanol can be converted to formaldehyde via methanol dehydrogenase MxaF, and a portion of the formaldehyde is automatically converted to methylene-THF, which is then converted to serine via GlyA and enters the serine cycle pathway. The acetyl-CoA produced in the serine cycle pathway can then enter the ethylmalonyl-CoA pathway. FIG. 1 shows various enzymes in the ethylmalonyl-CoA pathway, including: 3-ketothiolase (PhaA), acetoacetyl-CoA reductase (PhaB), crotonase (CroR), crotonyl-CoA carboxylase/reductase (Ccr), ethylmalonyl-CoA isomerase (Epi), ethylmalonyl-CoA mutase (Ecm), mesaconyl-CoA hydratase (Mcd), methylsuccinyl-CoA dehydrogenase (Msd), malyl-CoA/β-methylmalyl-CoA lyase (Mcl), propionyl-CoA carboxylase (Pcc), methylmalonyl-CoA mutase (Mcm), and succinyl-CoA synthetase (SucCD).

In other words, in methylotrophic bacteria, after methanol metabolism produces acetyl-CoA, the acetyl-CoA can be converted to succinate through the action of enzymes PhaA, PhaB, CroR, Ccr, Epi, Ecm, Mcd, Msd, Mcl, Pcc, Epi, Mcm, and SucCD.

Referring to FIG. 2, it can be seen that in the TCA cycle, succinate is converted to fumarate via succinate dehydrogenase (Sdh), thereby consuming the naturally produced succinate in methylotrophic bacteria.

In embodiments of the present disclosure, the activity of succinate dehydrogenase in TCA cycle of genetically modified strains can be reduced or inactivated by genetic engineering, such as gene editing technology, so that the genetically modified strains lose the ability to utilize succinate and start to accumulate succinate, and the produced succinate is excreted.

In some embodiments of the present disclosure, the naturally occurring methylotrophic bacterium Methylobacterium extorquens strain AM1 is used, and the succinate dehydrogenase subunits A and B (SdhAB) in the TCA cycle are knocked out through genetic engineering techniques. Such genetically modified strains can be cultured in complex medium. However, in the mineral medium, the strain is unable to convert succinate into malic acid, which leads to growth inhibition issues.

Using mineral medium as fermentation environment, compared to complex media, can reduce the cost of product purification, which helps achieve the industrial vision of converting methanol to succinate. Additionally, if malic acid needs to be added during the culture or fermentation process, it would further increase the culture cost.

In embodiments of the present disclosure, genetically engineered strains have been developed to address the growth inhibition issue caused by the inactivation or reduced activity of succinate dehydrogenase. Therefore, the genetically engineered strains are capable of producing high yields of succinate in mineral medium using methanol as the sole carbon source.

In some embodiments, the mineral medium includes water, a carbon source (i.e., methanol), inorganic salts (e.g., potassium, sodium, magnesium, calcium, bicarbonate plus carbonate, phosphate, sulfate, and chloride), a pure nitrogen salt (e.g., ammonium or urea), trace metals (e.g., iron, copper, zinc, manganese, cobalt, molybdenum, and optionally borate), optionally glycine betaine (also known as betaine), and optionally an antifoaming agent. The medium does not contain complex (also known as “rich”) nutrient sources, such as yeast extract, corn syrup, soybean hydrolysate, broth, casein hydrolysate, grains, legumes, or other “undefined” nutrient mixtures. Additionally, the medium may contain one or more pure chemical components to meet specific growth requirements (e.g., for auxotrophic strains or slow-growing strains) or to enhance a biochemical pathway. For example, some strains may require vitamins, or vitamins may promote the growth of some strains, so small concentrations of vitamins may be added to the medium.

In some embodiments, the components of the mineral medium used include: (NH₄)₂SO₄MgSO₄7H₂O, KH₂PO₄, Na₂HPO₄, CaCl₂), NaC₆H₃O₇·2H₂O, FeSO₄7H₂O, ZnSO₄7H₂O, MnCl₂·4H₂O, CuSO₄·5H₂O, CoCl₂6H₂O, H₃BO₃, Na₂WO₄·2H₂O and Na₂MoO₄·2H₂O.

In some embodiments, the medium contains about 0.5% v/v (volume/volume) to about 2% v/v methanol as the sole carbon source, such as 0.5% v/v, 1% v/v, 1.5% v/v, or 2% v/v methanol. Since methanol in the medium is metabolized or volatilized, methanol can be supplemented to the medium at regular intervals (e.g., about 8 to 16 hours) during the culture period.

In some embodiments, the genetically engineered microorganism is capable of growing and producing succinate without the addition of malic acid during the culture period. That is, during the initial and subsequent periods of culture, it is not necessary to add malic acid to the culture medium. In some embodiments, no other organic acids need to be added during the cultivation of the genetically engineered microorganisms for succinate production.

The following experimental examples provide genetically modified microorganisms and related experimental test results. The growth of the strains was quantified using OD₆₀₀ values from a spectrophotometer, and the succinate, its derivatives, or other byproducts in the culture were analyzed and quantified using high-performance liquid chromatography (HPLC).

### Experimental Example 1

In this experimental example, a succinate-producing methylotrophic bacterium was constructed to achieve direct methanol utilization and succinate production, enabling a one-step conversion of methanol to succinate. In this experimental example, Methylobacterium extorquens strain AM1 was selected as the host microorganism.

In this experimental example, strain M. extorquens AM1ΔcelABC was used as the base strain (TT-1), wherein ΔcelABC was introduced to reduce cellulose aggregation, which can hinder oxygen and nutrient exchange efficiency. The base strain TT-1 was further modified by knocking out the sdhAB gene, resulting in a strain with succinate production potential, M. extorquens AM1ΔcelABCΔsdhAB (also referred to as strain AR-1). This strain loses the ability to utilize succinate and can accumulate succinate.

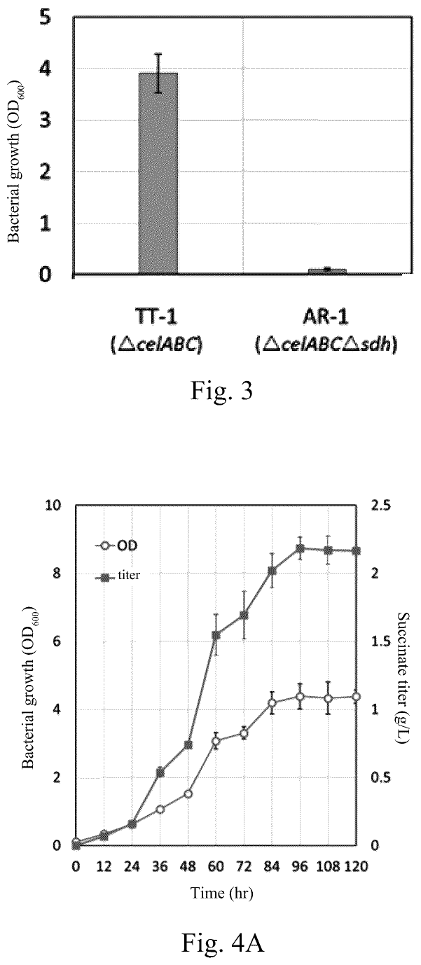

Subsequently, the base strain TT-1 and the sdhAB gene-deficient strain AR-1 were cultured in a complex medium (MC media) containing 60 mM succinate. As shown in FIG. 3, AR-1 did not grow after 24 hours, indicating the successful establishment of a methylotrophic bacterium strain having succinate production potential.

### Experimental Example 2

In this experiment, the sdhAB gene deficient strain AR-1 (M. extorquens AM1ΔcelABCΔsdhAB) was cultured respectively in a complex medium (CM) and mineral medium (MM) containing 1% (v/v) methanol to test the growth of the strain and the production of succinate.

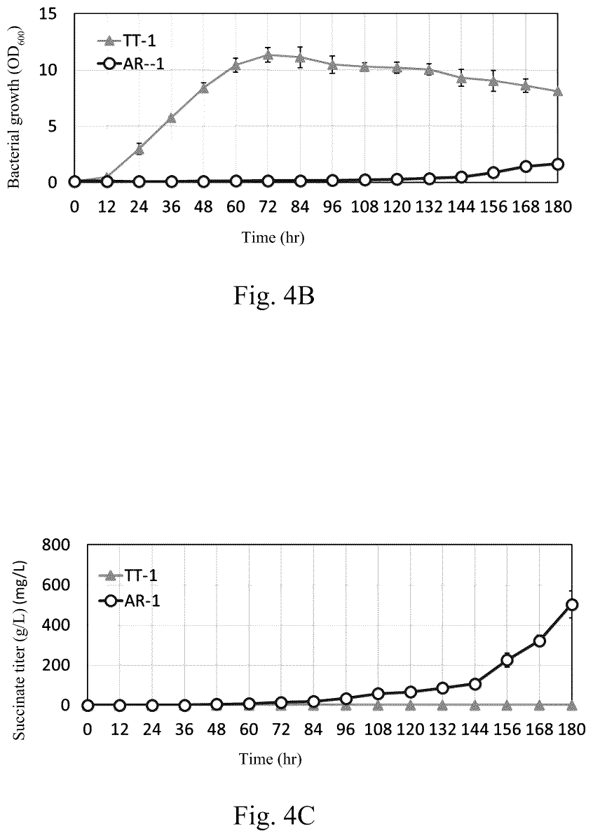

FIG. 4A shows the test results of culturing strain AR-1 in the complex medium. The growth and succinate titer of strain AR-1 in 20 mL of the complex medium were measured. Strain AR-1 entered the exponential growth phase (log phase) after 48 hours of culture in the complex medium and produced 2.1±0.1 g/L of succinate at the 96-hour time point, with a positive correlation between succinate titer and strain growth.

FIGS. 4B and 4C show the test results of culturing strains TT-1 and AR-1 in the mineral medium. FIG. 4B shows the growth of strains TT-1 and AR-1 in 20 mL of the mineral medium, and FIG. 4C shows the succinate titer of strains TT-1 and AR-1 in 20 ml of the mineral medium. The results show that in the mineral medium, the growth of strain AR-1 was significantly inhibited, requiring 180 hours of growth to reach an OD₆₀₀ value of 1.65 and produce 502±67 mg/L of succinate.

### Experimental Example 3

This experimental example involved adding intermediate metabolites to perform growth rescue tests on the strain AR-1. If the strain shows differences in growth rate after the addition of a specific intermediate metabolite, the metabolite can be used as a basis for analyzing the metabolic pathway bottleneck of strain AR-1.

The sdhAB gene-deficient strain AR-1 was cultured in a mineral medium containing 1% (v/v) methanol, and 1.18 g/L of acetate, 0.375 g/L of glycine, 0.37 g/L of glyoxylate, 1.76 g/L of pyruvate, 2.68 g/L of malate, 2.66 g/L of aspartate (ASP), and 2.92 g/L of α-ketoglutarate (α-KG) were respectively added to different culture vessels.

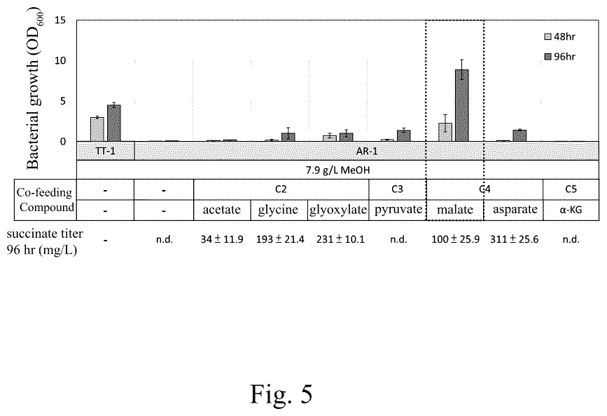

FIG. 5 shows the growth rescue test and succinate titer of sdhAB gene-deficient strain AR-1. In this figure, “n.d.” indicates that no succinate titer was detected. The results show that the addition of malate effectively restored the growth of strain AR-1. Under the condition of adding malic acid, the OD600 value of strain growth was about 2.2 at 48 hours, which was close to the OD600 value of control group (strain TT-1) of 2.9, but the growth was about 2 times higher than that of control group at 96 hours, and 100±26 mg/L succinate was produced.

### Experimental Example 4

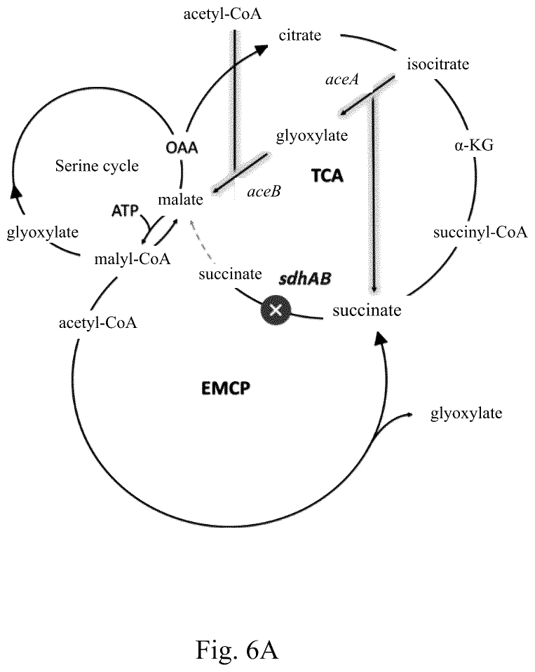

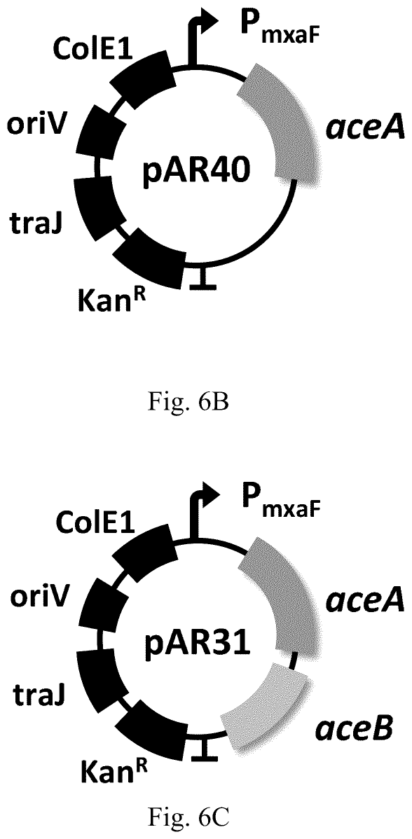

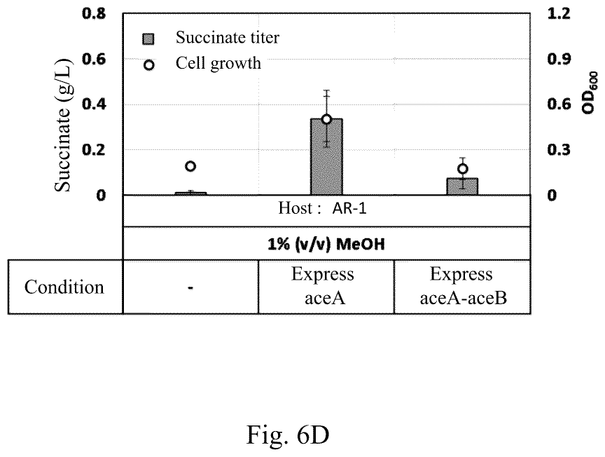

This experimental example involved overexpressing the genes of the glyoxylate shunt in the methylotrophic bacterium to restore the growth of the sdhAB gene-deficient strain AR-1 and increase succinate production. Referring to FIG. 6A, the serine cycle, TCA cycle, EMCP, and the glyoxylate shunt pathway of the methylotrophic bacterium are shown.

Since methylotrophic bacteria lack a natural glyoxylate pathway, in this experimental example, the genes encoding the enzymes of the glyoxylate pathway were introduced into the methylotrophic bacterium. Referring to FIG. 6A, the enzymes involved in the conversion of isocitrate to succinate, glyoxylate, and malate include isocitrate lyase (AceA) and malate synthase (AceB).

FIGS. 6B and 6C show the constructed expression plasmids. The expression plasmid pAR40 carries the aceA gene from E. coli, with the sequence shown as SEQ ID: 1 in the sequence listing. The expression plasmid pAR31 carries the aceA and aceB genes from E. coli, with the aceA gene sequence shown as SEQ ID: 1 and the aceB gene sequence shown as SEQ ID: 2 in the sequence listing. The P_{mxaF} fragment sequence is the methanol promoter of the methylotrophic bacterium. The oriV-traJ fragment sequence is the conjugation gene transfer gene of the methylotrophic bacterium AM1. The ColE1 fragment sequence is a commonly used replicon in E. coli. The Kan^{R} fragment sequence is the gene sequence for kanamycin resistance protein.

FIG. 6D shows the growth and succinate titer of strain AR-1 after transformation with plasmid pAR40 or pAR31, respectively, cultured in a mineral medium containing 1% (v/v) methanol for 48 hours. The results show that the expression of aceA alone can restore the growth of AR-1 to an OD₆₀₀ value of 0.5 and produce 0.3 g/L of succinate at 48 hours. Additionally, compared to the control group, the co-expression of aceA and aceB also can increase succinate titer.

### Experimental Example 5

This experimental example involved the natural evolution of the sdhAB gene-deficient strain AR-1 in a mineral medium to accelerate the growth rate of this strain, obtaining a methylotrophic variant strain capable of rapid growth in a mineral medium having methanol as the sole carbon source.

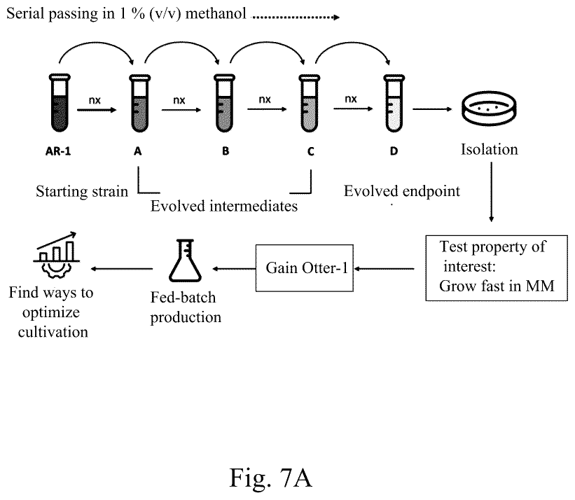

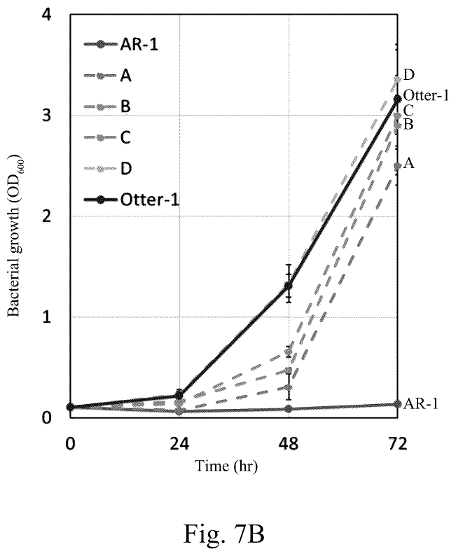

FIG. 7A shows the workflow of adaptive laboratory evolution (ALE). First, strain AR-1 was cultured in 2 mL of complex medium (MC media) containing 1% (v/v) methanol for 3 days of pre-culture, then 1% (v/v) of the strain cells were inoculated into another 2 mL mineral medium (MM media) containing 1% (v/v) methanol, serving as the starting point for evolution. After the bacterial strain reached a stable growth phase, transfer 1% (v/v) of the bacterial liquid to a new mineral medium and add methanol. By continuously subculturing and adapting the strain to utilize 1% (v/v) methanol, a faster-growing strain was isolated. During the process from pre-culture to subculture, the strains were maintained at 30° C. and 200 rpm. The growth rate was assessed by the change in OD₆₀₀ value per unit time during the rapid growth phase. During the passage period, a mixed solution of evolving variants was collected approximately every 1 to 1.5 months, and a 40% (v/v) glycerol-water mixture in a 1:1 ratio was used as a preservative and stored at −80° C. In total, four mixed solutions of variants, labeled A, B, C, and D, were obtained, along with a strain named Otter-1, which was isolated from a single colony of the D variant mixed solution.

FIG. 7B shows the growth curve of strain AR-1 and variant strains A, B, C, D, and Otter-1 in a mineral medium containing 1% (v/v) methanol. It can be observed that adopting adaptive laboratory evolution strategies can effectively accelerate the growth rate of the strains in mineral media, which is beneficial for shortening the production time of succinate. As described in Experimental Example 2, the growth of the strain may have a positive correlation with product generation. Additionally, using a mineral medium as the fermentation environment can reduce the costs of the product purification process compared to a complex medium. Therefore, the strain Otter-1, which has the fastest growth rate, is of significant importance for realizing the industrial vision of converting methanol into succinate.

### Experimental Example 6

In this experimental example, the differences in gene expression of the evolved variant strain Otter-1 were measured using quantitative reverse transcription polymerase chain reaction (RT-qPCR). The results can serve as a basis for analyzing the metabolic pathways of strain Otter-1 and for increasing succinate production. In this experimental example, several target genes related to the central metabolic pathway of methanol-utilizing bacteria were selected. Please refer to FIG. 2, which shows the metabolic steps associated with these target genes. The selected genes include: methanol dehydrogenase (mxaF), serine hydroxymethyl transferase (glyA), malate dehydrogenase (mdh), malyl-CoA lyase (mcl), acetoacetyl-CoA reductase (phaB), crotonyl-CoA carboxylase (ccr), and citrate synthase (gltA).

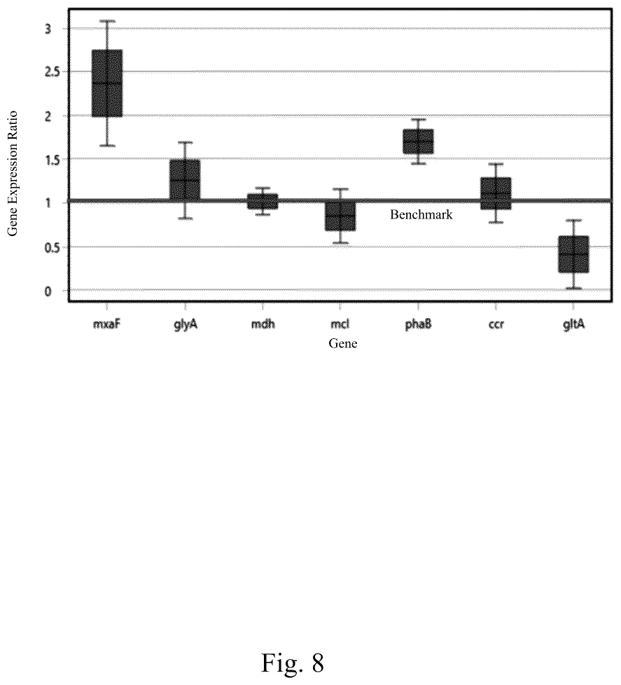

Referring to FIG. 8, the relative gene expression levels of RT-qPCR are shown, with the gene expression levels in strain AR-1 as the reference baseline. The results show that the expression of the mxaF gene increased by about 2.4 times, and the expression of the glyA gene increased by about 1.3 times, indicating that methanol metabolism was enhanced in variant strain Otter-1. Additionally, the expression of the phaB gene increased by 1.7 times, and the expression of the gltA gene significantly decreased. This indicates that variant strain Otter-1 produces succinate from methanol primarily through the ethylmalonyl-CoA pathway (EMCP).

The results indicate that strain Otter-1, obtained through adaptive evolution, overcomes the issue of growth inhibition by evolving enhanced methanol metabolism, inhibiting the TCA pathway, and increasing the carbon flux of the EMCP.

### Experimental Example 7

This experiment was conducted in conical flasks for fed-batch fermentation of succinate. At hour 0, 1% (v/v) methanol was added, and every 12 hours, an additional 1% (v/v) methanol was supplemented, along with adjustments to the pH of the medium to restore the medium to a pH of 7.

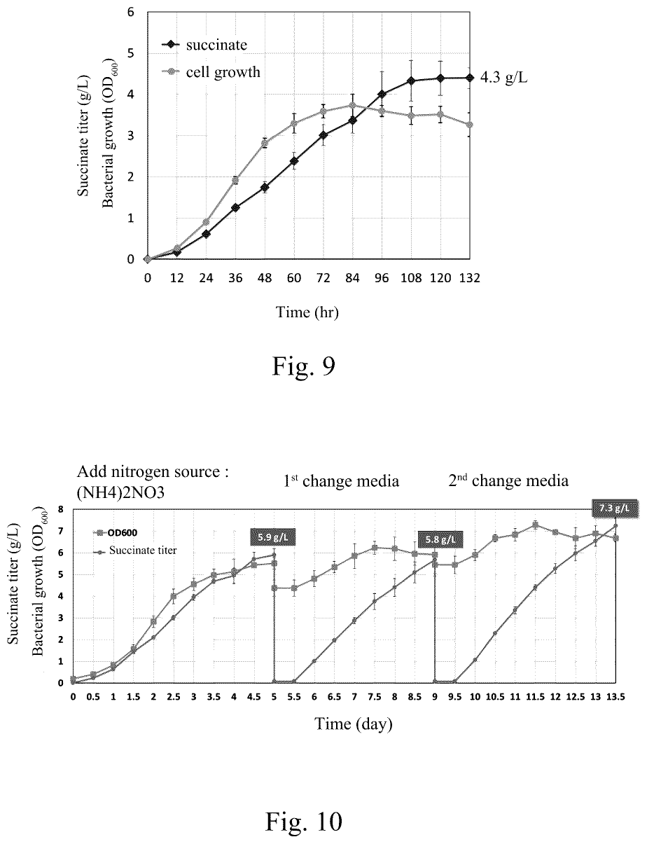

The results, as shown in FIG. 9, indicate that after 108 hours of culture, 4.3 g/L of succinate was produced, which shows high potential of methylotrophic bacteria for succinate production.

### Experimental Example 8

In this experiment, a nitrogen source was added during the fermentation process, and continuous batch fermentation was conducted to produce succinate.

A nitrogen source was added during the fed-batch fermentation of succinate to prolong the growth of the strain and accumulate higher concentrations of succinate.

During the testing period, on day 0, 1% (v/v) methanol was added to the mineral culture medium, and methanol was supplemented every half day (12 hours), along with the addition of 1 g/L of ammonium nitrate (NH₄)₂NO₃, and the pH of the culture medium was adjusted back to pH 7. As shown in FIG. 10, the addition of a nitrogen source can increase the yield of succinate to 5.9 g/L.

FIG. 10 also shows the subsequent replacement of fresh mineral medium, using a continuous batch fermentation method to allow the strain to regrow and produce succinate. The results showed that the strain achieved the highest succinate titer of 7.3 g/L after the second replacement of the culture medium. This result can serve as a strategy for modifying the culture conditions to increase succinate production. In other words, the strain can be continuously cultured for many days, and this fermentation cultivation method can effectively enhance the production capacity of succinate.

### Experimental Example 9

In this experiment, the variant strain Otter-1 was further characterized and the difference in gene sequence between the evolved variant Otter-1 and the base strain TT-1 was explored using Next Generation Sequencing (NGS). The following Table 1 and Table 2 show the results of next-generation sequencing analysis of the variant strain Otter-1, including the types of mutations that occurred in the chromosomes and the endogenous megaplasmid of the variant strain Otter-1. Table 1 shows the sites of base insertions or deletions, and Table 2 shows the sites of base substitutions. NC_012808.1 is the sequence code for the chromosome of methylotrophic bacterium AM1, while NC_012811.1 is the sequence code for the endogenous megaplasmid of methylotrophic bacterium AM1.

TABLE 1

| | | | Type | |
|---|---|---|---|---|
| Chromosome | Position | Encoded products | (INDEL) | Description |
| NC_012808.1 | 482893 | Flagellar hook-length | insertion | G -> GC |
| (Chromosome) | | control protein, FliK | | |
| | 2012134 | Hypothetical protein | deletion | GCCGTC -> G |
| | 2329710 | NTE family protein rssA | deletion | AG -> A |
| | 3037768 | Maltose alpha-D- | deletion | TC -> T |
| | | glucosyltransferase | | |
| | 3159070 | ATP-dependent helicase | deletion | CG -> C |
| | 4001526 | Glutathione | deletion | GCGTGC -> G |
| | 4001533 | S-transferase | deletion | GT -> G |
| | 4422401 | TonB-dependent | insertion | G -> GA |
| | 4422405 | receptor | deletion | GC -> G |
| | 4423386 | TonB-dependent | insertion | G -> GA |
| | 4423393 | receptor | insertion | C -> CTG |
| | 4755174 | Enoyl-CoA hydratase/ | deletion | GC -> G |
| | | isomerase family protein | | |
| | 4808561 | Hypothetical protein | deletion | ACCACGCGCC -> A |
| | 5018430 | MBL fold metallo-hydrolase | insertion | C -> CG |
| NC_012811.1 | 580985 | Hypothetical protein | insertion | G -> GC |
| (megaplasmid) | 770862 | Hypothetical protein | deletion | TG -> T |

TABLE 2

| Chromosome | Location | encoded products | Type (SNP) | describe |
|---|---|---|---|---|
| NC_012808.1 | 501077 | DUF4239 | substitution | G -> A |
| (Chromosome) | | domain-containing protein | | |
| | 1724528 | ABC transporter | | T -> G |
| | | ATP-binding protein | | |
| | 2012145 | Hypothetical protein | | C -> G |
| | 2050899 | TonB-dependent | | C -> G |
| | | siderophore receptor | | |
| | 2511236 | LEPR-XLL | | T -> C |
| | | domain-containing protein | | |
| | 2936238 | Hypothetical protein | | Hypothetical |
| | | | | protein |
| NC_012811.1 | 170244 | IS110 family transposase | | T -> C |
| (megaplasmid) | | ISMex12 | | |

Achieving higher levels of succinate production during fermentation process is desired. The following experimental example tested different cultivation conditions to further enhance the production yield of succinate biosynthesis by the strain Otter-1.

### Experimental Example 10

In the EMCP, two mutases (see FIG. 1), Ecm and Mcm, both use adenosylcobalamin (AdoB₁₂) as a cofactor to carry out reversible carbon skeleton rearrangement reactions. AdoB₁₂ is a type of vitamin B₁₂, and its molecular structure consists of a corrin ring with a cobalt ion (Co²⁺) at the center and a nucleotide composed of 5,6-dimethylbenzimidazole as the base. This experiment aims to investigate the effect of cobalt ions (Co²⁺) concentration on the growth of strains and the production of succinate. Cobalt chloride (CoCl₂) was added to a mineral medium without CoCl₂ at concentrations of 0, 0.4, 4, 12, 24, and 48 μM for testing, with 4 μM CoCl₂ serving as the control group. Additionally, the effect of adding vitamin B complex was also tested for comparison. Additionally, the effect of adding a vitamin B complex was tested for comparison. The test vitamin B complex included thiamine (vitamin B₁), riboflavin (vitamin B₂), nicotinamide (vitamin B₃), pantothenic acid (vitamin B₅), pyridoxine (vitamin B₆), biotin (vitamin B₇), folic acid (vitamin B₉), and cyanocobalamin (vitamin B₁₂).

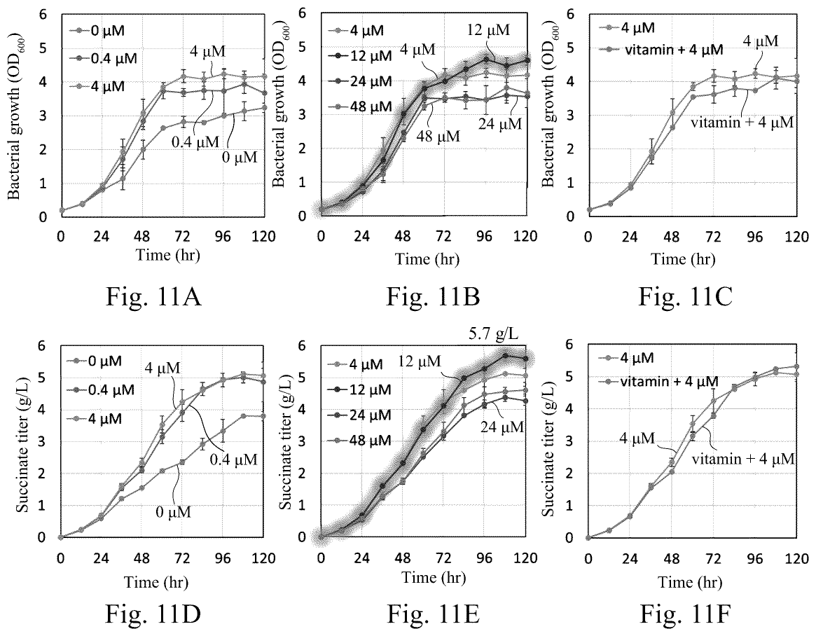

FIGS. 11A to 11F show the effects of different cobalt ion concentrations [Co²⁺] on the growth and succinate titer of strain Otter-1. FIGS. 11A and 11D compare the growth and succinate production at 0 and 0.4 μM cobalt ion concentrations [Co²⁺] with a baseline concentration of 4 M. FIGS. 11B and 11E show the growth and succinate titer at 12, 24, and 48 μM cobalt ion concentrations [Co²⁺]. FIGS. 11C and 11F show the growth and succinate titer after the addition of the vitamin B complex. In this experimental example, the initial cell density was OD₆₀₀ value of 0.1.

As shown in FIGS. 11A and 11D, the absence of cobalt ions in the environment severely affected strain growth and succinate titer. However, cobalt ion concentrations in the range of 0.4 M to 4 μM showed no significant effect.

FIGS. 11B and 11E show that when the cobalt ion concentration was increased to 12 μM, the succinate titer effectively increased to 5.7 g/L. However, further increasing the cobalt ion concentration to 24 UM and 48 μM inhibited growth and succinate titer.

FIGS. 11C and 11F show the results of adding the vitamin B complex, which did not show significant differences compared to the control group. This may be because the vitamin B₁₂ in the vitamin B complex is cyanocobalamin rather than adenosylcobalamin, and the Ecm and Mcm enzymes prefer to use adenosylcobalamin.

In summary, based on the test results of this experimental example, in some embodiments, the quantification experiments for succinate used a mineral medium containing 12 UM cobalt ions. In some embodiments, the cobalt ion concentration in the mineral medium is less than 20 μM, for example, in the range of 0.4μ M to 20μ, preferably in the range of 1μ M to 18μ, or 2μ M to 16μ M.

### Experimental Example 11

In the above experimental examples of fermentative succinate production using the Strain Otter-1 mentioned above, we found that a total of approximately 6 to 7 g/L of NaOH needed to be added to maintain the pH of the culture environment at 7 for 120 hours. However, this could potentially lead to an excessive accumulation of sodium ions (Na⁺), which in turn may inhibit the growth of the strain and the production of succinate.

In this experimental example, different concentrations of NaCl (0, 2, 5, 10, 15, 30, and 60 g/L) were respectively added to the culture medium of the strain Otter-1 at 0 hours to test the effect of different sodium ion concentrations on the growth of the strain. Strain growth was measured over 48 hours.

As shown in FIG. 12, when the NaCl concentration reached 5 g/L, it inhibited about 50% of strain growth. This indicates that one of the reasons for the growth stagnation of the strain Otter-1 in the late growth stage may be the toxicity caused by the accumulation of too many sodium ions.

To address the issue of sodium ion accumulation, this experiment selected other common neutralizing agents, KOH and NH₄OH, for testing. During the culture period, 0.3 to 0.4 g/L of KOH or 0.5 to 0.6 g/L of NH₄OH was added every 12 hours. The initial cell density was an OD₆₀₀ value of 0.1. The growth of strain Otter-1 and succinate titer were measured every 12 hours, and the culture period was 108 hours.

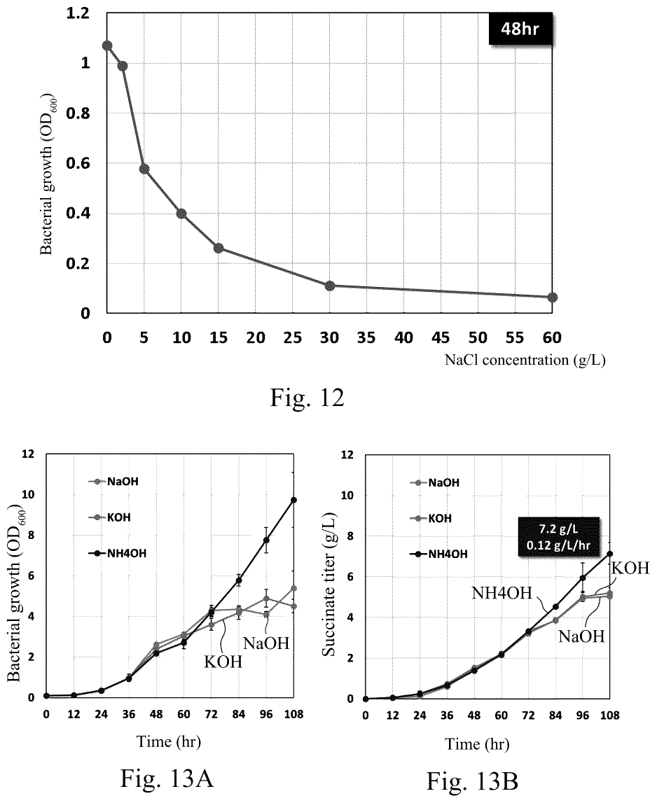

As shown in FIGS. 13A and 13B, whether in terms of strain growth or succinate titer, the results of using KOH to adjust the pH were similar to those of using NaOH, possibly due to excessive metal ions (K⁺). However, the test group using NH₄OH showed an increase in strain growth of about OD₆₀₀ value of 2 at 108 hours, with succinate titer reaching 7.2 g/L and accumulating at a rate of about 0.12 g/L/hour. The reason may be that NH₄_{⁺} can be utilized by the strain as a nitrogen source at suitable concentrations, and there is less risk of excessive metal ion accumulation.

According to the test results of this experimental example, in some embodiments thereafter, the concentration of NaCl in the culture medium is less than 5 g/L. In some embodiments, NH₄OH can be added as a neutralizer for the culture medium, for example, the NH₄OH concentration in the culture medium may be 6 to 7 g/L.

### Experimental Example 12

The choice of fermentation vessel can affect the amount of methanol that volatilizes, which would be detrimental to analyzing the yield of succinate produced by the strains. Therefore, in this experimental example, the optimal cultivation conditions mentioned above (including a mineral medium with 12 μM cobalt and pH adjusted with NH₄OH) were combined, and the fermentation flasks used had angular bodies that could create turbulence, with two different types of caps tested. The bottle caps in the control group are made of white polypropylene (PP) plastic, also known as “white PP caps”; the bottle caps in the test group have a sponge, also referred to as “sponge head caps.” In this experimental example, no additional NH₄NO₃ was added, as NH₄OH was already added to supplement the nitrogen source.

In this experimental example, both the white PP cap and the sponge head cap are oxygen-permeable. Using a cap having a sponge not only reduces methanol volatilization but also reduces water vapor volatilization, possibly due to the porous material of the sponge head. Therefore, the gas exchange rate of the flask with the sponge head cap is lower than that of the flask with the white PP cap.

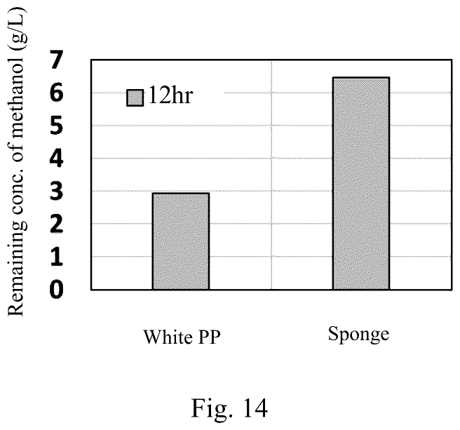

FIG. 14 is a bar chart showing the remaining amount of methanol in the methanol aqueous solution in the flasks of the control group and the experimental group after 12 hours.

The effects of these two caps on the fermentation of strain Otter-1 were then tested. For the control group (white PP cap) and the test group (sponge head cap), methanol was added every 12 hours and the pH was adjusted to 7; for the test group (sponge head cap), methanol was no longer added after 108 hours. The initial cell density was OD₆₀₀ value of 0.5. The initial methanol concentration was 7.9 g/L. Additionally, methanol concentration changes, succinate production, and strain growth were monitored every 12 hours.

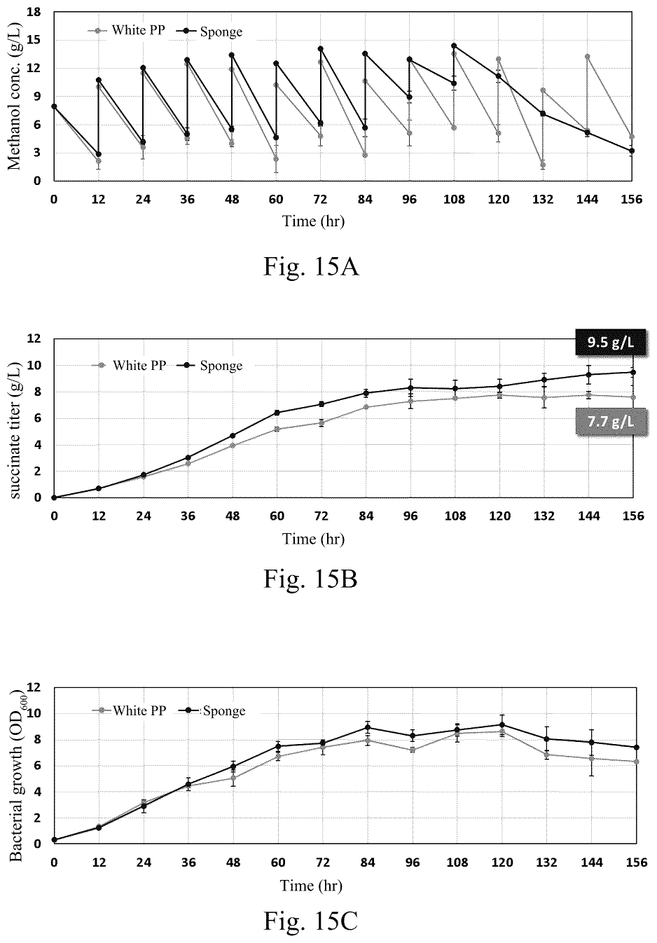

As shown in FIGS. 15A to 15C, there is no significant difference in the methanol concentration changes between the control group using white PP caps and the test group with sponge head caps during the early fermentation stage (before 84 hours), with both groups showing approximately 6 to 7 g/L every 12 hours. This data includes the amount of methanol that has evaporated and the actual amount metabolized by the strains. It is possible that the strains metabolize methanol more quickly in the early stage due to their growth requirements, resulting in a smaller impact from methanol evaporation, making the effect of methanol volatilization less observable. However, in the later stages of fermentation, the methanol concentration changes in the test group (sponge head cap) gradually decreased, with a change of 2 to 3 g/L every 12 hours. This indicates that as the strain's growth stagnated, its methanol metabolism rate also slowed (see FIG. 15C). In contrast, the control group with the white PP cap continued to show a methanol variation of about 6 to 7 g/L every 12 hours, but the growth of the strain and the production of succinate did not increase, similar to the test group with the sponge cap, indicating the severity of methanol volatilization.

It is noted that under the condition of methanol concentration not lower than 2 g/L, simply replacing the bottle cap resulted in a difference in succinate production between the control group and the test groups (see FIG. 15B). The production for the white PP cap group and the sponge head cap group was 7.7 g/L and 9.5 g/L, respectively.

In some embodiments, a fermentation tank is used to culture the strain on a larger scale and produce succinate. In some embodiments, the fermentation tank includes means to reduce methanol volatilization. In some embodiments, the exhaust port of the fermentation tank is equipped with a condensation device, such as a cooling coil or a condensation tower, to condense and recover volatilized methanol, thereby reducing methanol emissions. In some embodiments, the fermentation temperature may be reduced (e.g., controlled at 20 to 30° C.) to reduce volatilization. In some embodiments, the tank pressure may be increased within an appropriate range to reduce the methanol escape rate. In some embodiments, physical adsorption and filtration may also be used to capture methanol molecules.

The genetically engineered strain of the present disclosure can overcome the growth inhibition caused by the inactivation of succinate dehydrogenase and the use of methanol as the sole carbon source in mineral medium. In some embodiments, genetic editing techniques or adaptive evolution are used to cause further mutations in the succinate dehydrogenase-deficient methylotrophic bacterium, resulting in a strain that produces succinate using methanol as the sole carbon source.

Since the strain Otter-1, obtained through adaptive evolution, has overcome the issue of growth inhibition by enhancing methanol metabolism, inhibiting the TCA cycle, and increasing the carbon flux of EMCP, these changes can be utilized as a strategy for constructing genetically engineered microorganisms.

In some embodiments, genetic editing techniques or adaptive evolution can be used to make the succinate-producing strain have increased methanol metabolism pathway activity, reduced TCA pathway activity, and increased carbon flux through the ethylmalonyl-CoA pathway.

In some embodiments, genetic editing techniques or adaptive evolution are used to make the genetically engineered microorganism have: enhanced expression of at least one gene involved in the methanol metabolism pathway; enhanced expression of at least one gene involved in the serine cycle; reduced expression of at least one gene involved in the TCA cycle pathway; and enhanced expression of at least one gene involved in the ethylmalonyl-CoA pathway. In some embodiments, the at least one gene involved in the methanol metabolism pathway includes a methanol dehydrogenase gene (mxaF). In some embodiments, the at least one gene involved in the serine cycle includes a serine hydroxymethyltransferase gene (glyA). In some embodiments, the at least one gene involved in the TCA cycle pathway includes a citrate synthase gene (gltA). In some embodiments, the at least one gene involved in the ethylmalonyl-CoA pathway includes: an acetoacetyl-CoA reductase gene (phaB) or a crotonyl-CoA carboxylase/reductase gene (ccr), or a combination thereof.

In some embodiments, the host of the genetically engineered microorganism is a methylotrophic bacterium, containing endogenous or exogenous genes located in the host cell. In some embodiments, recombinant technology can be used to construct plasmids carrying and expressing genes involved in the methanol metabolism pathway, the serine cycle pathway, and/or the ethylmalonyl-CoA pathway. In some embodiments, genetic editing techniques can be used to reduce the expression of TCA pathway genes in the genetically engineered strain.

The genetically engineered microorganisms provided by embodiments of the present disclosure, through the modification of the metabolic pathways of methylotrophic bacteria, can efficiently produce succinate using mineral medium with methanol as the sole carbon source. Therefore, the biosynthesis of succinate can be easily industrialized, reducing culture costs and being environmentally friendly.

Although the present disclosure has been disclosed in embodiments and examples above, it is not intended to limit the present disclosure. Any person skilled in the art can make various modifications and changes without departing from the spirit and scope of the present disclosure. Therefore, the protection scope of the present disclosure shall be defined by the appended claims.

What is claimed is:

1. A genetically engineered microorganism for producing succinate, comprising:
a host cell, wherein the host cell is derived from a methylotrophic bacterium, the host cell has an ethylmalonyl-CoA pathway, an activity of succinate dehydrogenase (Sdh) in the methylotrophic bacterium is reduced or inactivated, and the genetically engineered microorganism is capable of growing and producing succinate using methanol as a sole carbon source.

2. The genetically engineered microorganism for producing succinate of claim 1, further comprising:
at least one heterologous gene encoding an enzyme of glyoxylate cycle or an enzyme converting isocitrate to glyoxylate and succinate.

3. The genetically engineered microorganism for producing succinate of claim 2, wherein the at least one heterologous gene is located in a plasmid.

4. The genetically engineered microorganism for producing succinate of claim 2, wherein the at least one heterologous gene comprises: a gene encoding isocitrate lyase (aceA), a gene encoding malate synthase (aceB), or a combination thereof.

5. The genetically engineered microorganism for producing succinate of claim 1, wherein the genetically engineered microorganism is obtained through adaptive laboratory evolution.

6. The genetically engineered microorganism for producing succinate of claim 5, wherein during the adaptive laboratory evolution, performing serial passage using a mineral medium having methanol as a sole carbon source.

7. The genetically engineered microorganism for producing succinate of claim 5, wherein the genetically engineered microorganism is a strain of Methylobacterium extorquens Otter-1, with deposit number BCRC 911257.

8. The genetically engineered microorganism for producing succinate of claim 1, wherein the genetically engineered microorganism has:
enhanced expression of at least one gene involved in methanol metabolism pathway;
enhanced expression of at least one gene involved in serine cycle;
reduced expression of at least one gene involved in tricarboxylic acid (TCA) cycle pathway; and
enhanced expression of at least one gene involved in the ethylmalonyl-CoA pathway.

9. The genetically engineered microorganism for producing succinate of claim 8, wherein:
the at least one gene involved in the methanol metabolism pathway comprises a methanol dehydrogenase gene (mxaF);
the at least one gene involved in the serine cycle comprises a serine hydroxymethyltransferase gene (glyA);
the at least one gene involved in the TCA cycle pathway comprises a citrate synthase gene (gltA); and
the at least one gene involved in the ethylmalonyl-CoA pathway comprises: an acetoacetyl-CoA reductase gene (phaB) or a crotonyl-CoA carboxylase/reductase gene (ccr), or a combination thereof.

10. A method for producing succinate, comprising:
cultivating a strain for producing succinate, comprising: cultivating the genetically engineered microorganism for producing succinate of claim 1 in a mineral medium, wherein a sole carbon source in the mineral medium is methanol.

11. The method for producing succinate of claim 10, wherein the cultivating of the strain for producing succinate is performed using fed-batch fermentation or continuous batch fermentation.

12. The method for producing succinate of claim 10, further comprising: supplementing a nitrogen source to the mineral medium.

13. The method for producing succinate of claim 12, wherein the nitrogen source is (NH₄)₂NO₃ or NH₄OH.

14. The method for producing succinate of claim 10, wherein the mineral medium comprises vitamin B₁₂.

15. The method for producing succinate of claim 10, wherein the mineral medium comprises NaCl, and a concentration of the NaCl is less than 5 g/L.

16. The method for producing succinate of claim 10, wherein the mineral medium comprises NH₄OH and NaCl as neutralizers.

17. A method for making a genetically engineered microorganism, comprising:
performing a first gene editing operation to reduce or inactivate activity of succinate dehydrogenase (Sdh) in a methylotrophic microorganism, wherein the methylotrophic microorganism comprises an ethylmalonyl-CoA pathway; and
performing a second gene editing operation or an adaptive evolution operation to obtain a strain for producing succinate using methanol as a sole carbon source.

18. The method for making a genetically engineered microorganism of claim 17, wherein through the second gene editing operation, the strain for producing succinate expresses a heterologous enzyme of glyoxylate cycle or an enzyme converting isocitrate to glyoxylate and succinate.

19. The method for making a genetically engineered microorganism of claim 17, wherein through the second gene editing operation or the adaptive evolution operation, the strain for producing succinate has increased methanol metabolism pathway activity, reduced TCA cycle pathway activity, and increased carbon flux through the ethylmalonyl-CoA pathway.

20. The method for making a genetically engineered microorganism of claim 17, wherein the adaptive evolution operation comprises serial passaging the methylotrophic microorganism using a mineral medium having methanol as a sole carbon source.
